(19) United States
(12) Patent Application Publication
(10) Pub. No.: US 2020/0333329 A1
(43) Pub. Date: Oct. 22, 2020

(54) TARGETED CYTOKINE BLOCKADES FOR CAR-T THERAPY

(71) Applicant: H. LEE MOFFITT CANCER CENTER AND RESEARCH INSTITUTE, INC., Tampa, FL (US)

(72) Inventor: Marco L. Davila, Tampa, FL (US)

(21) Appl. No.: 16/610,279

(22) PCT Filed: May 1, 2018
(86) PCT No.: PCT/US18/30526

§ 371 Date: Nov. 1, 2019

Related U.S. Application Data
(60) Provisional application No. 62/492,753, filed on May 1, 2017.

(51) Int. Cl. G01N 33/53; G01N 33/68; A61K 35/17

(52) U.S. Cl. CPC G01N 33/53; G01N 33/6863; G01N 2800/52; G01N 2333/52; A61K 35/17

(57) ABSTRACT

Disclosed are compositions and methods for detecting the cytokines upregulated in cytokine release syndrome and neurotoxicities associated with CAR-T cell toxicity and methods of targeted treatment of cytokines associated with the same.

This application claims the benefit of U.S. Provisional Application No. 62/492,753, filed on May 1, 2017 which is incorporated herein by reference in its entirety.

### I. BACKGROUND

T cell therapy has significant potential as a cancer therapy because T cells can expand in large numbers to eradicate high volume disease, can traffic throughout disparate areas of the body to eradicate residual tumor sites, and can endow patients with long-lived tumor immunity. However, major disadvantages that limit the utility of adoptive T cell therapy include the MHC restriction of antigen presentation to T cell receptors (TCR) MHC downregulation as a mechanism of immune escape, and the lengthy production time required to create a sufficient number of tumor-specific T cells. A major advance for adoptive T cell therapy that addressed these limitations is the chimeric antigen receptor (CAR), which is a single chain variable fragment (scFv) fused to the activation domains of a TCR. Antigen-specificity is encoded by the scFv, which is derived from the antigen-binding domains of an antibody.

As a “living drug” tumor-specific T cells can induce long-term immunity. The promise of T cell therapy was realized when CR rates of 88% was demonstrated after treating relapsed B cell acute lymphoblastic leukemia (B-ALL) patients with CD19-targeted CAR T cells. These results suggest the first FDA-approved gene-modified cell therapy is on the horizon. However, significant challenges can preclude application of this technology to a wider patient population. A new set of toxicities including the cytokine release syndrome (CRS) and neurotoxicity, have also been associated with CAR T cells. Severe adverse events (SAE≥grade 3), represented by CRS or neurologic toxicity, occur in about 30-40% of patients. Medical interventions with cytokine blockade have been used with success in some patients but the choice of when and what agent to use is decided by clinical judgement, which is guided in part by unreliable biomarkers. What are needed are assays for identifying the cytokines that are adherently upregulated following CAR-T therapy and inform and guide the management of severe CAR T cell toxicities.

### II. SUMMARY

Disclosed are methods related to treating CAR T-cell toxicity. In one aspect, disclosed herein are methods of treating a subject with CAR-T cell toxicity comprising: a) obtaining a tissue sample from a subject; b) performing an immunoassay that measures cytokines on the tissue sample; c) obtaining a cytokine profile for the subject; and d) adjusting therapy based on the cytokine profile; wherein a subject with elevated IFNγ relative to a control is treated with a steroid; a subject with elevated TNFα relative to a control is treated with anti-TNF blocking treatment; a subject elevated IL-1 relative to a control is treated with anti-IL-1 blocking treatment, a subject with elevated IL-6 relative to a control is treated with anti-IL-6 blocking treatments; a subject with multiple elevated cytokines relative to a control is treated with one or more of a steroid, an anti-IL-6 blocking treatment, an anti-IL-1 blocking treatment, and an anti-TNF blocking treatment; and a subject with no elevated cytokines relative to a control does not receive anti-cytokine therapy.

In one aspect, the CAR-T cell toxicity can be cytokine release syndrome or neurotixicity.

Also disclosed are methods of any preceding aspect, wherein the immunoassay used to detect upregulation of cytokines is selected from the group consisting of enzyme linked immunosorbent assays (ELISAs), Ella™, enzyme linked immunospot assays (ELispot), radioimmunoassays (RIA), immunobead capture assays. Western blotting, dot blotting, gel-shift assays, intracellular cytokine stain, immunohistochemistry, protein arrays, and multiplexed bead arrays.

Also disclosed are methods of any preceding aspect, wherein the tissue sample from the subject is whole blood, serum, Peripheral blood mononuclear cells (PBMC), bone marrow, or cerebrospinal fluid (CSF).

Also disclosed are methods of any preceding aspect, wherein the control is a tissue sample from the subject obtained prior to start of CAR T cell therapy.

Disclosed herein are also methods of detecting elevated cytokines in a subject with CAR T cell toxicity comprising: a) obtaining a tissue sample from a subject; and b) performing an immunoassay that measures cytokines on the tissue sample; wherein an increase in a cytokine level in a subject relative to a control indicates elevated cytokines.

Also disclosed are methods of any preceding aspect, wherein the immunoassay used to detect upregulation of cytokines is selected from the group consisting of enzyme linked immunosorbent assays (ELISAs), Ella™, enzyme linked immunospot assays (ELIspot), radioimmunoassays (RIA), immunobead capture assays, Western blotting, dot blotting, gel-shift assays, intracellular cytokine stain, immunohistochemistry, protein arrays, and multiplexed bead arrays.

Also disclosed are methods of any preceding aspect, wherein the tissue sample from the subject is whole blood, serum, Peripheral blood mononuclear cells (PBMC), bone marrow, or cerebrospinal fluid (CSF).

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, and 1F show the increase in cytokine expression for patients receiving CAR-T cell therapy. FIG. 1A shows the increase in IFN-γ expression. FIG. 1B shows the increase in TNF-α expression. FIG. 1C shows the increase in IL-1 expression. FIG. 1D shows the increase in Il-6 expression. FIG. 1E shows the cytokine expression profile of patients with no increase in cytokine expression. FIG. 1F shows the cytokine expression profile of patients with an increase in cytokine expression for all cytokines measured.

FIG. 2 shows a comparison of cytokine levels measured by luminex vs. the Ella. Serum samples from patients with B-ALL treated with CD19-targeted CAR T cells were collected and measured by luminex or Ella technologies. The serum levels are directly compared and evaluated by a correlation coefficient to evaluate the similarities.

FIG. 3 shows that patients with B-ALL treated with CD19-targeted CAR T cells have serum collected and evaluated for cytokine elevations. The max-fold change for cytokines are compared in patients with severe or no cytokine release syndromes.

FIG. 4 shows daily monitoring a patient's cytokine levels on an Ella. A patient with a B cell malignancy treated with CD19-targeted CAR T cells has their serum collected daily and measured on the Ella. IFNg, TNFα, IL1b, and IL6 are measured in the serum.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Efficacious re-induction of CR's by CAR T cells is associated with a unique set of clinical signs and symptoms of a massive inflammatory disorder. Shortly after infusion of CAR T cells, patients develop high-grade fevers that sometimes progress with hypotension and respiratory distress. Coincident with these toxicities is a large increase of numerous cytokines so this disorder has been classified as a cytokine release syndrome (CRS). It is likely that the CRS is related to the widespread activation of a large number of tumor-specific T cells considering that similar toxicities have been reported with blinatumomab and anti-CD28 antibodies. While some cytokines, such as IFNγ, IL6, and IL10 are commonly increased after CAR T cell infusion there is no consistent pattern of cytokine upregulation from patient to patient, which is due to the individualized nature of the CAR T cell therapy. The CRS can progress to severe and life-threatening, ultimately requiring intensive medical management. Grading schemes have been developed to differentiate CRS that require close monitoring to episodes that require more aggressive interventions. The Memorial Sloan Kettering Cancer Center (MSKCC) group identified severe CRS based on the presence of fevers, cytokine elevations, and clinical signs of severe toxicity, such as hypotension requiring pressors or hypoxia requiring mechanical ventilation. A collaborative group of CAR T cell investigators developed a revised scheme based on the requirement of medical interventions to support patients so Grade 1 is self-limiting, while Grade 4 is life-threatening. Comparison of the grading schemes indicates that Grades 3-5 from Lee et al would be classified as severe CRS according to the Davila et al criteria.

Severe CRS was reported by MSKCC in 7 out of 16 patients, by University of Pennsylvania (UPENN) in 8 out 30 patients, by the National Cancer Institute (NCI) in 6 out of 21 patients, and by the Fred Hutchinson Cancer Research Center (FHCRC) in 7 out of 30 patients. CRS has even resulted in fatal toxicities, although this has been relatively infrequent. Only 2 deaths out of 97 B-ALL patients treated with CD19-targeted CAR T cells have been attributed to CRS. For most patients mild to moderate CRS (i.e. Grades 1-2) is self-limiting and requires only supportive care, but in severe cases medical intervention is required. Cytokine-directed therapy and steroids are the mainstay of CRS management. The MSKCC group treated 4 patients with tocilizumab and 3 with steroids, the UPENN group treated 9 with tocilizumab and 6 with steroids, the NCI treated 2 with tocilizumab and 2 with both steroids and tocilizumab, and FHCRC treated 7 patients with tocilizumab and 3 with steroids. Cytokine-directed therapy includes tocilizumab and etanercept, which block the IL6-Receptor (IL6R) and TNF signaling, respectively. Tocilizumab is more widely employed since IL6 commonly increases during rapid progression of CRS. However, a valid concern is if these interventions could limit the efficacy of CAR T cell eradication of leukemia. In fact, it has been reported that steroids inhibit CAR T cell expansion and reduce the durability of remissions. Due to a limited understanding of the mechanism of these toxicities efforts prior to the work herein were unable to develop targeted supportive therapies that can ameliorate toxicities without reducing anti-leukemia efficacy.

All clinical trials evaluating CD19-targeted CART cells for B-ALL have reported neurologic toxicities. These toxicities include word-finding difficulty, aphasia, encephalopathy, obtundation, and generalized seizures. The exact mechanism for neurotoxicity is unknown. Neurotoxicities and CRS are considered to be separate toxicities since they can occur at disparate times during the clinical course. UPENN reported that 6 of 13 cases of neurologic complications occurred after CRS had completely resolved. However, neurologic toxicities are related to T cell activation since similar complications develop in patients treated with blinatumomab. Indeed, CAR T cells can be detected in the cerebrospinal fluid (CSF) after treatment with peak serum cytokine levels correlating with severity of neurotoxicity. This suggests en masse activation of the CAR T cells, directly or indirectly, endows the cells with the ability to traverse the blood brain barrier (BBB). However, presence of B-ALL in the Central Nervous System (CNS) may also contribute to neurologic toxicities since CAR T cells are increased in the CNS of patients with residual disease versus those patients without residual CNS disease. In terms of severe (≥Grade 3) neurotoxicities the FHCRC reported 15 out of their 30 treated patients and MSKCC reported 6 of 17 patients developed this level of complication. The NCI reported 6 out of their 21 patients and UPENN reported 13 of their 30 treated patients developed any grade neurotoxicity. Management includes prophylaxis and medical interventions. Many patients were given seizure prophylaxis medications but currently there is no evidence that prophylaxis has reduced the number of neurologic complications and/or severity. Similar to CRS the medical interventions for neurologic toxicities are tocilizumab and steroids. While nearly all patients ultimately respond to steroids it is uncertain if tocilizumab ameliorates the neurologic toxicities because this antibody is unable to cross the BBB. However, tocilizumab may still provide benefit by reducing inflammation, which could impede CAR T cells ability to cross the BBB. What the market lacks is a targeted approach to treating CAR T cell toxicity.

To meet the need for targeted anti-cytokine therapy to treat CAR T cell toxicity, disclosed are methods related to treating CAR T-cell cytotoxity. In one aspect, disclosed herein are methods of treating a subject with CAR-T cell toxicity comprising: a) obtaining a tissue sample from a subject; b) performing an immunoassay that measures cytokines on the tissue sample; c) obtaining a cytokine profile for the subject; and d) adjusting therapy based on the cytokine profile; wherein a subject with elevated IFNγ relative to a control is treated with a steroid; a subject with elevated TNFα relative to a control is treated with anti-TNF blocking treatment; a subject elevated IL-1 relative to a control is treated with anti-IL-1 blocking treatment, a subject with elevated IL-6 relative to a control is treated with anti-IL-6 blocking treatments; a subject with multiple elevated cytokines relative to a control is treated with one or more of a steroid, an anti-IL-6 blocking treatment, an anti-IL-1 blocking treatment, and an anti-TNF blocking treatment; and a subject with no elevated cytokines relative to a control does not receive anti-cytokine therapy.

In one aspect, the CAR-T cell toxicity being treated in the disclosed methods or can be cytokine release syndrome or neurotoxicity.

It is understood and herein contemplated that the methods of treating CAR T cell toxicity incorporate the detection of elevated cytokines in a subject with CAR T cell toxicity. Thus, in one aspect, disclosed herein are methods of detecting elevated cytokines in a subject with CAR T cell toxicity comprising: a) obtaining a tissue sample from a subject; and b) performing an immunoassay that measures cytokines on the tissue sample; wherein an increase in a cytokine level in a subject relative to a control indicates elevated cytokines.

The disclosed methods of treatment and methods of detection utilize immunoassays for the detection of elevated cytokine levels relative to a control. The control can be any negative control that can be used as a baseline for a subject including a tissue sample from the subject obtained prior to onset of CAR T cell therapy.

It is understood and herein contemplated that the methods of treating CAR T Cell toxicity or detecting elevated cytokines in a subject with CAR T cell toxicity disclosed herein comprise the detection of cytokines in tissue samples from the subject being treated. As contemplated herein tissue samples can comprise any biological sample from which cytokines can be measured, including, but not limited to whole blood, serum, Peripheral blood mononuclear cells (PBMC), bone marrow, cerebrospinal fluid (CSF), tissue biopsy, lung lavage, partial or whole splenectomy, and lymph nodes.

The cytokines being measured can be any cytokine associated with a neurotoxicity or cytokine release syndrome falling within CAR T cell toxicity, including, but not limited to IFNγ, TNFα, IL-6, IL-1 (including IL-1β), Il-2, IL-5, IL-8, IL-10 and IL-13. Cytokines can be measured by any immunological assay known in the art including, but not limited to enzyme linked immunosorbent assays (ELISAs), microfluidic host cell protein assay (such as, for example Simple Plex™ run on Ella™), enzyme linked immunospot assays (ELIspot), radioimmunoassays (RIA), immunobead capture assays, Western blotting, dot blotting, gel-shift assays, intracellular cytokine stain, immunohistochemistry, protein arrays, and multiplexed bead arrays. Thus, in one aspect, disclosed herein are methods of treating CAR T cell toxicity or detecting elevated cytokine levels in a subject with CAR T cell toxicity, wherein the immunoassay used to detect upregulation of cytokines is selected from the group consisting of enzyme linked immunosorbent assays (ELISAs), Ella™, enzyme linked immunospot assays (ELIspot), radioimmunoassays (RIA), immunobead capture assays, Western blotting, dot blotting, gel-shift assays, intracellular cytokine stain, immunohistochemistry, protein arrays, and multiplexed bead arrays.

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELSAs), Ella™, Enzyme-Linked Immunospot Assay (ELISPOT), radioimmunoassays (RIA), radioimmune precipitation assays (RIPA), immunobead capture assays (such as, magnetic bead capture), Western blotting, dot blotting, gel-shift assays, Flow cytometry, protein arrays, multiplexed bead arrays, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 15 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethyirhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FI; Bodipy FL ATP; Bodipy FI-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson −; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.18; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 143™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type’ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1 LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue LysoSensor Green; LysoSensor Yellow/Blue; Mag Green; Magdala Red (Phloxin B); Mag-Fura Red Mag-Fura-2; Mag-Fura-5; Mag-Indo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-1 PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2, Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green Rhodamine Phallicidine; Rhodamine; Phalloidine; Rhodamine Red; Rhodamine WT; Rose Bengal; R-phycocyanine; R-phycoeythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARFL; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60 SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™ Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodamineIsoThioCyanate; True Blue; Tru Red; Ultralite, Uranine B; Uvitex SFC; wt GFP; WW 781; X-Rhodamine; XRTC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the aspect including, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing. SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10 MW for known samples, and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, N G, High resolution two-dimensional electrophoresis of human plasma proteins, Proc. Natl. Acad. Sci. 74:5421-5425 (1977), Omstein, L., Disc electrophoresis, L. Ann. N.Y. Acad. Sci. 121:321349 (1964), each of which is herein incorporated by reference in its entirety for teachings regarding electrophoresis methods. Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Omstein L., Disc electrophoresis—I: Background and theory, Ann. NY Acad. Sci. 121:321-349 (1964), and Matsudiara, P T and D R Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promegacom/faq/gelshfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and immune bead capture assays (for example magnetic bead capture).

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, D-galactosidase, ribonuclease, urease, catalase, malate dehydrogenase, staphylococcal nuclease, asparaginase, yeast alcohol dehydrogenase, alpha.-gly cerophosphate dehydrogenase, triose phosphate isomerase, glucose-6-phosphate dehydrogenase, glucoamylase and acetylcholinesterase.

Variations of ELISA techniques are know to those of skill in the art including methods and assays utilizing microfluidics. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed. ELISAs have certain features in common, such as coating, incubating or binding, washing to remove non-specifically bound species, and detecting the bound immunecomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding agent or a secondary binding agent in conjunction with a labeled third binding agent.

In one aspect, the immunoassay can be a microfluidic host cell protein detection assay. Such assays are known in the art and can include the use of a glass-polydimethilsiloxane microfluidic network and an array of photosensors, micropatterned aptamer modified electrodes, Ab spots printed onto poly(ethylene glycol) (PEG) hydrogel-coated glass slides, AlphaPlex, AlphaLisa, and Simple Plex ELLA. For example, the microfluidic host cell protein immunoassay can comprise Simple Plex™. Simple Plex™ is a two component assay that measures host-cell proteins (HCP) comprising a disposable cartridge and a microfluidic analyzer (such as, for example, Ella™). Simple Plex run on Ella (the assay and machine being used interchangeably herein as Ella), solves a problem faced by ELISAs that can take 4-6 hours to complete. Ella provides 4-5 logs of sensitivity in 1 hours. Like an ELISA, Ella measures HCP, but unlike ELISAs, Ella is an automated system and therefore eliminates variability introduced from inconsistent washing and antibody use. In an Ella, the sample is drawn into the system and split into four parallel isolated microfluidic channels. This is where Ella differs from other approaches such as AlphaPlex and AlphaLisa which accomplish multiplex immunoassays by using a common acceptor bead to bind multiple analytes and antibodies in a single reaction prior to incubation. By contrast in Ella, each channel is isolated and has a single-plex immunoassay for a single analyte run in triplicate and thus avoids antibody cross-reactivity. In this automated assay, the system is primed, then the sample is pumped through the system cartridge. After the sample has been evenly distributed, the sample is incubated and following a set incubation period the circuit is washed. Next analyte specific antibodies are individually pumped through one of the isolated circuits. Following the administration of the antibody, the system is washed to remove unbound antibody and a detectable fluor is pumped through the system to bind to the antibodies for detection. Following a further wash, the fluor is excited with a 631 nm laser and read by a detector (such as, for example, a CCD camera). Ella allows for multiplex results without the concerns of cross-reactivity are typical of a multiplex ELISA and which result in decreased sensitivity. Accordingly, in one aspect, disclosed herein are methods of treating a subject with CAR-T cell toxicity comprising: a) obtaining a tissue sample from a subject; b) performing an immunoassay that measures cytokines on the tissue sample; c) obtaining a cytokine profile for the subject; and d) adjusting therapy based on the cytokine profile; wherein the immunoassay comprises an automated system; wherein the immunoassay comprises at least one antibody for detection of at least one analyte; wherein the detection of each analyte is isolated from the detection of other analytes; wherein the immunoassay is performed by applying a biological sample to a system, drawing the sample into the isolated chambers and incubating the sample in the isolated chambers, simultaneously and independently contacting the analyte in each isolated chamber with a single anti-analyte antibody, and detecting the bound antibody (such methods can further comprise adding a detectable agent if a non-labeled antibody is used and it is understood that any detection method known in the art can be used such as x-ray, radiation, and fluorescence detection); wherein a subject with elevated IFNγ relative to a control is treated with a steroid; a subject with elevated TNFα relative to a control is treated with anti-TNF blocking treatment; a subject elevated IL-1 relative to a control is treated with anti-IL-1 blocking treatment, a subject with elevated IL-6 relative to a control is treated with anti-IL-6 blocking treatments; a subject with multiple elevated cytokines relative to a control is treated with one or more of a steroid, an anti-IL-6 blocking treatment, an anti-IL-1 blocking treatment, and an anti-TNF blocking treatment; and a subject with no elevated cytokines relative to a control does not receive anti-cytokine therapy.

Enzyme-Linked Immunospot Assay (ELISPOT) is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

“Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA and/or ELIspot, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzothiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic beads and other microbeads. While microdrops of protein delivered onto planar surfaces are the most familiar format, alternative architectures include CD centrifugation devices based on developments in microfluidics (Gyros, Monmouth Junction, N.J.) and specialized chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include colour coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging. New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma. St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staphylococcus aureus protein A (Affibody, Bromma, Sweden). ‘Trinectins’ based on fibronectins (Phylos, Lexington, Mass.) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colors. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford Conn.).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BDT Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

As stated above, the disclosed detection methods and treatment methods can be used direct a targeted therapeutic treatment in a subject with CAR T cell toxicity. In one aspect, the treatment can comprise the therapeutic blockade of the elevated cytokine rather than a general suppression of all cytokines with merely anti-IL-6 receptor blockade or a general steroid. For example, a subject with elevated IFN-γ could not be treated with an anti-IFNγ antibody but could receive a steroid; a subject with elevated TNF-α can be treated with an anti-TNFα antibody or other blockade, such as the anti-TNF antibodies infliximab, adalimumab, certolizumab, golimumab or the TNF receptor fusion protein Etanercept; a subject with elevated IL-1 relative to a control can be treated with an anti-IL-1β or IL-1 receptor (IL-1R) antibodies such as, for example, the anti-IL-IP antibody Canakinumab or the IL-1 receptor antagonist anakinra; a subject with elevated IL-6 relative to a control can be treated with an anti-IL-6 or anti-IL-6R antibodies such as, for example the anti-IL-6R antibodies tocilizumab and sarilumab and/or the anti-IL6 antibodies olokizumab, elsilimomab, and siltuximab; a subject with elevated IL-6 relative to a control can be treated with an anti-IL-6 or anti-IL-6R. It is understood and herein contemplated that where a subject has more than one elevated cytokine relative to a control any combination of two or more cytokine blockades or steroids may be used to treat the subject.

1. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include antibodies for the detection of IFNγ, TNFα, IL-6, IL-1 (including IL-1β), Il-2, IL-5, IL-8, IL-10 and IL-13.

### B. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Validation of a Daily Cytokine Monitoring System for CRS

Disclosed herein is the rapid detection of CRS and guidance for cytokine-blocking intervention. Monitoring of cytokines associated, directly or indirectly, with EM CAR T cells facilitates the diagnosis of severe CRS and guide intervention. Evidence supporting this observation come from clinical data demonstrating elevation of cytokines that are secreted by T cells or secreted by other cells recruited by T cells.

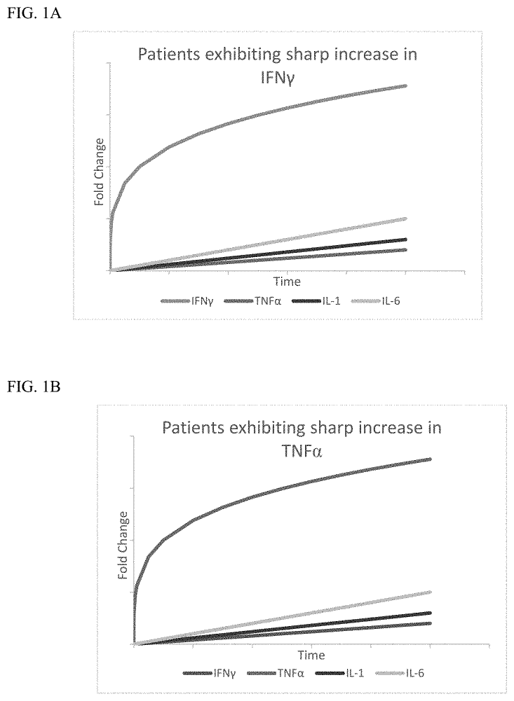

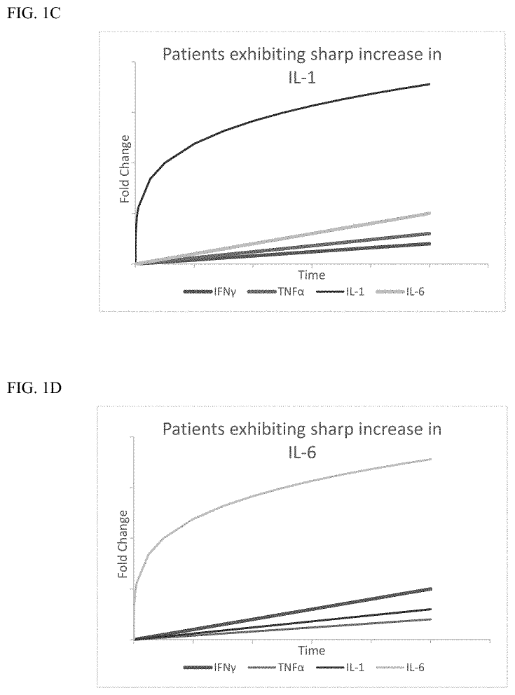

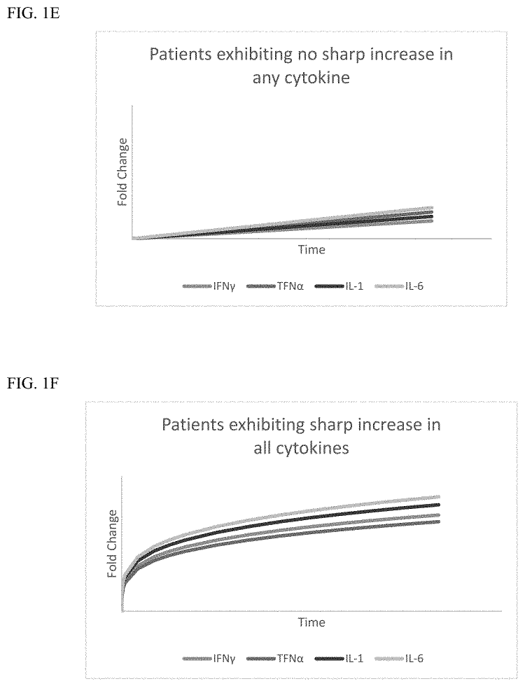

The rationale is providing objective cytokine data rapidly to physicians supports informed clinical decision-making and reduce morbidity and mortality associated with CAR T cells. Serum is collected from patients treated with CD19-targeted CAR T cells and measure cytokine expansion. Equipment can be used that measures cytokine levels in 1 hour. IFNγ, TNFα, IL1, IL6 are compared and the equivalence of the standard 2-day multiplex and rapid 1-hour assay can be confirmed as shown in FIG. 1. In the scenario associated with FIG. 1A, IFNγ is upregulated while none of the cytokines are upregulated. This patient may experience toxicities but they cannot be intervened with cytokine blocking agents. They would require steroids. In the scenario associated with FIG. 1B, TNFα is upregulated while none of the other cytokines are upregulated. To ameliorate toxicities this patient would be treated with anti-TNF blocking treatments. In the scenario associated with FIG. 1C, IL-1 is upregulated while none of the other cytokines are upregulated. To ameliorate toxicities this patient would be treated with anti-IL1 blocking treatments (e.g. ankinra). In the scenario associated with FIG. 1D, IL-6 is upregulated while none of the other cytokines are upregulated. To ameliorate toxicities this patient would be treated with anti-IL6 blocking treatments (e.g tocilizumab). In the scenario associated with FIG. 1E, none of the cytokines are upregulated and this patient would be unlikely to experience cytokine-associated toxicities. In the scenario associated with FIG. 1F, all cytokines are upregulated and can be treated with any cytokine blocking agents and/or steroids.

IL1, IL6, and TNFα can be blocked with commercially available antibodies. However, tocilizumab is most frequently used because the C-reactive protein biomarker can be measured daily and is correlated with IL6. Patients treated with tocilizumab are identified and grouped according to 75-fold change in IL6, which can be a threshold barrier associated for severe CRS. On determination that can be made is if patients with <75-fold change in IL6 treated with tocilizumab take longer for CRS to resolve than patients with ≥75-fold increase of IL6 treated with tocilizumab. Similarly, a determination of whether patients had ≥75-fold increases of TNFα or IL1 that could have been alternatively blocked.

a) Methods

(1) Patient Identification & Selection

Patients can be screened based on their enrollment and planned treatment with T cells. Up to 1 month prior to conditioning chemotherapy baseline samples are collected, which can be pre-infusion product or samples collected when patients are classified as having Grade 0-2 CRS or Grade 0-2 Neurotoxicity. Post T cell infusion samples are collected and this study includes fresh, non-cryopreserved CSF, and/or BM, and/or blood and/or serum obtained from 100 patients treated with adoptively transferred T cells. Patient PHI information is stored on a password-protected computer and the database file is password protected to maximize security of PHI. This file is accessible by the study investigators.

(2) Biospecimen Description

The following type of tissue specimens from patients are analyzed for immune phenotype, target killing, cytokine production, and pathogenesis. The sample numbers and volumes being request are necessary to allow a sufficient number to be analyzed (Table 1).

Serum—up to 10 mL of freshly isolated serum is collected from blood or BM. One baseline sample is collected and up to 28 daily samples are collected post CAR T cell infusion. These samples can be collected as extra samples as part of clinically indicated blood monitoring.

Peripheral blood mononuclear cells (PBMC) via phlebotomy—up to 40 mL of freshly isolated samples. One baseline sample and up to 7 samples are collected during episodes of severe CRS (≥grade 3). These samples can be collected as extra samples as part of clinically indicated blood monitoring.

Bone Marrow (BM)—up to 20 mL of freshly isolated BM is collected from bone marrow aspirations of the pelvis while the patient is anesthetized. One baseline sample and up to 2 samples are collected during episodes of severe CRS (≥grade 3). These samples can be collected as extra samples as part of a clinically indicated BM aspiration.

Cerebrospinal Fluid (CSF)—up to 10 mL of freshly isolated samples are collected from lumbar punctures performed with anesthesia. One baseline sample and up to 2 samples are collected during episodes of sever neurotoxicity (≥grade 3). These samples can be collected as extra samples as part of a clinically indicated lumbar puncture.

All these samples can be collected as extra samples as part of clinically indicated laboratory tests. No phlebotomy or procedures are performed solely for this protocol. Samples are prepared to allow cryopreservation in a locked liquid N₂ tank in MRC 2068. Samples are stored up to 5 years after cryopreservation.

TABLE 1
Sample collection

| | Baseline (1 sample | Daily (1 sample | | |
|---|---|---|---|---|
| | up to 30 days prior | collected daily | | Neuro- |
| | to CAR T infusion | up to 28 days | CRS ≥3 | toxicity ≥ |
| | or from Grade 0-2 | post CAR T | (up to 7 | 3 (up to 7 |
| Sample | toxicity) | infusion) | samples) | samples) |
| Serum | YES | YES | Already | Already |
| | | | collected | collected |
| PBMC | YES | NO | YES | NO |
| BM | YES | NO | YES | NO |
| CSF | YES | NO | NO | YES |
| Serum | YES | YES | Already | Already |
| | | | collected | collected |
| PBMC | YES | NO | YES | NO |
| BM | YES | NO | YES | NO |
| CSF | YES | NO | NO | YES |

(3) Serum Preparation:

Blood or BM are centrifuged for 10 minutes and serum are decanted, aliquoted, and cryopreserved. Serum are used to evaluate cytokine levels.

(4) Leukocyte Isolation:

Leukocytes are isolated from PBMC, BM, or CSF. Cells are enumerated after washes in PBS and partitioned for in vitro or in vivo assays.

(5) Flow Cytometry:

Cells are incubated with fluorochrome-conjugated antibodies for evaluation of activation, memory, naïve, and exhaustion markers.

(6) Ex Vivo Cytotoxic T Cell Stimulation and Target Killing:

These assays are performed with T cell fractions in triplicate. T cells are stimulated with artificial antigen-presenting cells that express human CD19. Activation is measured by target killing, cell expansion, and cytokine production.

(7) Cell Sorting:

CAR T cells are sorted and either injected into NSG mice implanted with B cell leukemias or submitted to the Moffitt Genomics Core Facility.

(8) Statistics

The primary endpoint for aim 1 is 30-day survival in mice injected with T cells from diseased patients compared to mice injected with control T cells. The sample size of 100 is determined to allow sufficient power for comparison, while allowing drop-offs. It is expected that about 40% of the patients cannot be collected because the patients may die, withdraw consent, or be potentially too sick to continue with the protocol. This allows the collection of 30 vs 30 in experimental and control group respectively. It is anticipated that the one-month survival rate for mice injected with T cells from the disease patients to be <50%, and the one-month survival rate of mice injected with healthy donors to be 80%. 30 vs 30 yields 80% power in comparing 80% vs 50% one-month survival rate based on one-sided test at significance level 0.05. There is no interim analysis planned for this study.

Descriptive statistical summaries as well as parametric (t test) or non-parametric statistical test (Wilcoxon Rank Sum Test) are performed as appropriate comparing experimental vs control group for immune phenotyping, tumor killing, and cytokine increases.

### 2. Example 2: CRS Detection and Monitoring Using Simple Plex™ on an Ella™ Machine

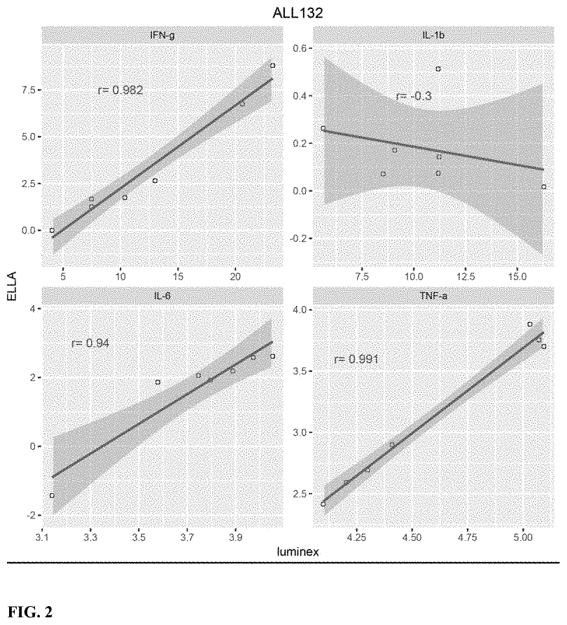

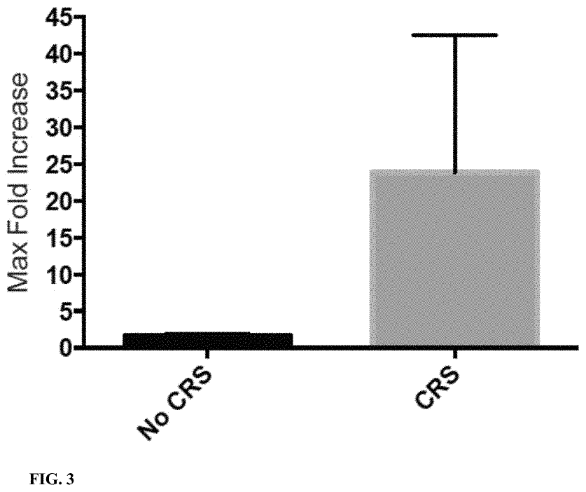

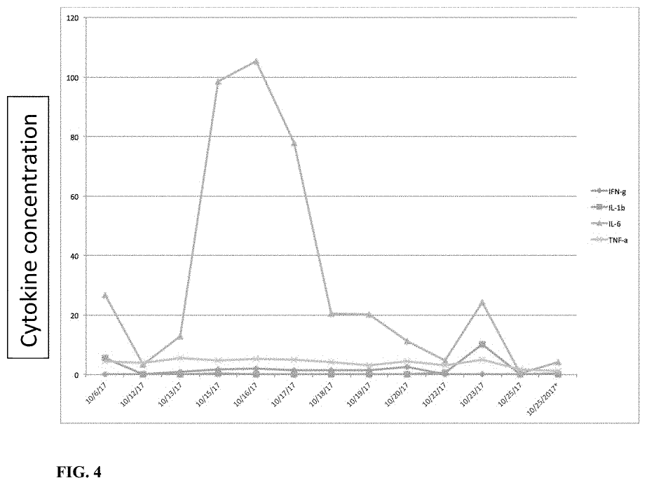

Simple Plex™ is a two component assay that measures host-cell proteins (HCP) comprising a disposable cartridge and a microfluidic analyzer (such as, for example, Ella™) Recognizing that a system such as Ella which obtains results 6× faster than conventional means; has minimal variability; reduced cross-reactivity and therefore has increased sensitivity would be optimal for monitoring and treating CRS, Applicants set to determine if Ella could be used to detect and monitor CRS and if so, guide intervention. First Applicants validated Ella by running it in parallel to the same detection using a Luminex (FIG. 2). Serum samples from patients with B-ALL treated with CD19-targeted CAR T cells were collected and measured by luminex or Ella technologies Applicants observed a direct correlation between Luminiex and Ella for the detection of TNF-α, IFN-γ, IL-1b, and IL-6. Next, Applicants determined that Ella could distinguish between B-ALL patients with and without CRS following CAR-T cell treatment (FIG. 3). Specifically, patients with B-ALL were treated with CD19-targeted CART cells and had serum collected and evaluated for cytokine elevations. Finally, Ella was validated in its ability to monitor cytokine levels in a patient overtime (FIG. 4). In particular, B-ALL patients were treated with CD19-targeted CART cells. Serum was collected and measured for TNF-α. IFN-γ, IL-1b, and IL-6 using ELLA.

### C. References

Brentjens R J, Davila M L, Riviere I, et al. CD19-targeted T cells rapidly induce molecular remissions in adults with chemotherapy-refractory acute lymphoblastic leukemia. Sci Transl Med 20135(177):177ra138.
Davila M L, Brentjens R. Wang X, Riviere I, Sadelain M. How do CARs work?: Early insights from recent clinical studies targeting CD19. Oncoimmunology. 2012; 1(9):1577-1583.
Davila M L, Riviere I. Wang X, et al. Efficacy and toxicity management of 19-28z CAR T cell therapy in B cell acute lymphoblastic leukemia. Sci Transl Med 2014; 6(224):224ra225.
Grupp S A, Kalos M, Barrett D, et al. Chimeric antigen receptor-modified T cells for acute lymphoid leukemia. N Engl J Med 2013; 368(16):1509-1518.
Kalos M. Levine B L, Porter D L, et al. T cells with chimeric antigen receptors have potent antitumor effects and can establish memory in patients with advanced leukemia. Sci Tansl Med. 2011:3(95):95ra73.
Lee D W, Gardner R, Porter D L, et al. Current concepts in the diagnosis and management of cytokine release syndrome. Blood. 2014:124(2):188-195.
Lee D W, Kochenderfer J N, Stetler-Stevenson M, et al. T cells expressing CD19 chimeric antigen receptors for acute lymphoblastic leukaemia in children and young adults: a phase 1 dose-escalation trial. Lancet. 2015; 385(9967):517-528.
Maude S L, Frey N. Shaw P A, et al. Chimeric antigen receptor T cells for sustained remissions in leukemia. N Engl J Med 2014:371(16):1507-1517.
Suntharalingam G, Perry M R, Ward S, et al. Cytokine storm in a phase 1 trial of the anti-CD28 monoclonal antibody TGN1412. N Engl J Med 2006; 355(10):1018-1028.
Teachey D T, Rheingold S R, Maude S L, et al. Cytokine release syndrome after blinatumomab treatment related to abnormal macrophage activation and ameliorated with cytokine-directed therapy. Blood. 2013:121(26):5154-5157.
Topp M S, Gokbuget N, Zugmaier G, et al. Phase II Trial of the Anti-CD19 Bispecific T Cell-Engager Blinatumomab Shows Hematologic and Molecular Remissions in Patients With Relapsed or Refractory B-Precursor Acute Lymphoblastic Leukemia. J Clin Oncol. 2014; 32(36):4134-4140.
Turtle C J, Hanafi L A, Berger C, et al. CD19 CAR-T cells of defined CD4+:CD8+ composition in adult B cell ALL patients. J Clin Invest. 2016.

What is claimed is:

1. A method of treating a subject with CAR-T cell toxicity comprising:
a) obtaining a tissue sample from a subject;
b) performing an immunoassay that measures cytokines on the tissue sample;
c) obtaining a cytokine profile for the subject; and
d) adjusting therapy based on the cytokine profile:
wherein a subject with elevated IFNγ relative to a control is treated with a steroid; a subject with elevated TNFα relative to a control is treated with anti-TNF blocking treatment; a subject elevated IL-1 relative to a control is treated with anti-IL-1 blocking treatment, a subject with elevated IL-6 relative to a control is treated with anti-IL-6 blocking treatments; a subject with multiple elevated cytokines relative to a control is treated with one or more of a steroid, an anti-IL-6 blocking treatment, an anti-IL-1 blocking treatment, and an anti-TNF blocking treatment; and a subject with no elevated cytokines relative to a control does not receive anti-cytokine therapy.

2. The method of claim 1, wherein the CAR-T cell toxicity is cytokine release syndrome.

3. The method of claim 1, wherein the CAR-T cell toxicity is neurotoxicity.

4. The method of claim 1, wherein the immunoassay used to detect upregulation of cytokines is selected from the group consisting of enzyme linked immunosorbent assays (ELISAs), Ella, enzyme linked immunospot assays (ELIspot), radioimmunoassays (RIA), immunobead capture assays, Western blotting, dot blotting, gel-shift assays, intracellular cytokine stain, immunohistochemistry, protein arrays, and multiplexed bead arrays.

5. The method of claim 1, wherein the tissue sample from the subject is whole blood, serum, Peripheral blood mononuclear cells (PBMC), bone marrow, or cerebrospinal fluid (CSF).

6. A method of detecting elevated cytokines in a subject with CAR T cell toxicity comprising:
a) obtaining a tissue sample from a subject; and
b) performing an immunoassay that measures cytokines on the tissue sample;
wherein an increase in a cytokine level in a subject relative to a control indicates elevated cytokines.

7. The method of claim 6, wherein the immunoassay used to detect upregulation of cytokines is selected from the group consisting of enzyme linked immunosorbent assays (ELISAs), Ella, enzyme linked immunospot assays (ELIspot), radioimmunoassays (RIA), immunobead capture assays, Western blotting, dot blotting, gel-shift assays, intracellular cytokine stain, immunohistochemistry, protein arrays, and multiplexed bead arrays.

8. The method of claim 7, wherein the tissue sample from the subject is whole blood, serum, Peripheral blood mononuclear cells (PBMC), bone marrow, or cerebrospinal fluid (CSF).
